(19) United States
(12) Patent Application Publication
(10) Pub. No.: US 2025/0207208 A1
(43) Pub. Date: Jun. 26, 2025

(54) Markers for Iron Deficiency Chlorosis

(71) Applicant: BASF Agricultural Solutions US LLC, Research Triangle Park, NC (US)

(72) Inventors: Jieqing Ping, Morrisville, NC (US); Keith Franklin Rincker, Seymour, IL (US); Raymond Matthew Kennett, Seward, NE (US)

(73) Assignee: BASF Agricultural Solutions US LLC, Research Triangle Park, NC (US)

(21) Appl. No.: 18/989,929

(22) Filed: Dec. 20, 2024

Related U.S. Application Data
(60) Provisional application No. 63/613,770, filed on Dec. 22, 2023.

(51) Int. Cl. C12Q 1/6895; A01H 1/00

(52) U.S. Cl. CPC C12Q 1/6895; A01H 1/122; C12Q 2600/13; C12Q 2600/156

(57) ABSTRACT

One embodiment relates to markers that are associated with resistance to Iron Deficiency Chlorosis in soybean and which can be used for producing soybean lines with improved resistance to Iron Deficiency Chlorosis. Another embodiment relates to methods and compositions for identifying, selecting and/or producing a soybean plant or germplasm having resistance to Iron Deficiency Chlorosis using genetic markers and the markers themselves. Further embodiments, include selecting and/or producing a soybean plant or germplasm having resistance to Iron Deficiency Chlorosis by any of the methods disclosed herein, are also provided.

### CROSS-REFERENCE TO RELATED APPLICATIONS

This application claims priority to U.S. Provisional Application No. 63/613,770 filed on Dec. 22, 2023, the entire contents of which are hereby incorporated by reference.

### SUBMISSION OF SEQUENCE LISTING

The Sequence Listing associated with this application is filed in electronic format via Patent Center and hereby incorporated by reference into the specification in its entirety. The name of the “xml” file containing the Sequence Listing is 231701US02_SEQLISTING_St26.xml. The size of the xml file is 6 KB, and the xml file was created on Nov. 29, 2024.

### BACKGROUND

Soybean, Glycine max (L.) Merr., is an important and valuable field crop. A continuing goal of soybean plant breeders is to develop stable, high yielding soybean cultivars that are agronomically sound. To accomplish this goal, the soybean breeder must select and develop soybean plants that have traits that result in superior cultivars. To accomplish this goal, the breeder must efficiently select and develop plants, which includes using molecular techniques, that have the traits that result in superior cultivars. All references cited herein are incorporated by reference in their entirety.

The foregoing examples of the related art and limitations related therewith are intended to be illustrative and not exclusive. Other limitations of the related art will become apparent to those of skill in the art upon a reading of the specification.

### SUMMARY

The following embodiments and aspects thereof are described and illustrated in conjunction with products and methods, which are meant to be exemplary and illustrative, not limiting in scope. In various embodiments, one or more of the above-described problems have been reduced or eliminated, while other embodiments are directed to other improvements.

One embodiment relates to markers that are associated with improved tolerance to Iron Deficiency Chlorosis (“IDC”) in soybean and which can be used for producing soybean lines with improved tolerance to Iron Deficiency Chlorosis.

Another embodiment relates to methods and compositions for identifying, selecting and/or producing a soybean plant or germplasm having improved tolerance to Iron Deficiency Chlorosis.

Another embodiment relates to selecting and/or producing a soybean plant or germplasm having improved tolerance to Iron Deficiency Chlorosis by any of the methods disclosed herein.

Another embodiment relates to a method for developing an Iron Deficiency Chlorosis tolerant soybean plant, wherein the method comprises applying marker enhanced selection to detect one or more polymorphisms, wherein said one or more polymorphisms are selected from the nucleotide polymorphisms of SEQ ID NOs:1-2.

Another embodiment relates to a method of producing a soybean plant that has improved tolerance to Iron Deficiency Chlorosis as compared to a control plant, wherein the method comprises: (a) isolating a nucleic acid from a soybean plant; (b) detecting in the nucleic acid, the presence of a genetic marker that is associated with improved tolerance to Iron Deficiency Chlorosis, wherein said genetic marker is selected from SEQ ID NOs:1-2; (c) selecting a first soybean plant based on the presence of the marker associated with improved tolerance; (d) crossing a second soybean plant with said first soybean plant, wherein the second soybean plant does not comprise in its genome the marker associated with improved tolerance to Iron Deficiency Chlorosis; (e) producing seed from said crossing; and (f) selecting a soybean plant grown from said seed that has improved tolerance to Iron Deficiency Chlorosis and comprises the genetic marker associated with improved tolerance to Iron Deficiency Chlorosis. Another further embodiment relates to a soybean plant produced by said method, wherein the plant comprises the genetic marker associated with improved tolerance to Iron Deficiency Chlorosis.

Another embodiment relates to a method of further comprising the step of backcrossing the plants produced from step (f).

One embodiment comprises markers that are associated with resistance to Iron Deficiency Chlorosis in soybean and which can be used for producing soybean lines with improved resistance. In another embodiment, a method for selecting one or more Glycine max plants comprising resistance to Iron Deficiency Chlorosis is provided. A further embodiment provides where the method comprises, (i) obtaining nucleic acids from a sample soybean plant or its germplasm; (ii) detecting one or more markers that indicate resistance to Iron Deficiency Chlorosis, and (iii) indicating resistance to Iron Deficiency Chlorosis.

Another embodiment further comprises methods and compositions for identifying, selecting and/or producing the one or more soybean plants or germplasm having resistance to Iron Deficiency Chlorosis.

Another embodiment relates to selecting and/or producing a soybean plant or germplasm having resistance to Iron Deficiency Chlorosis by any of the methods disclosed herein.

Another embodiment relates to a method of determining the genotype of a soybean plant, wherein said method comprises obtaining a sample of nucleic acids from the soybean plant and detecting in the nucleic acids, a plurality of polymorphisms, wherein said plurality of polymorphisms correspond to the insertion or deletion identified in any one or more of SEQ ID NOs:1-2.

Another embodiment relates to a method for developing an Iron Deficiency Chlorosis resistant soybean plant, wherein the method comprises applying marker enhanced selection to detect one or more polymorphisms, wherein said one or more polymorphisms are selected from the insertion or deletion of any one or more of SEQ ID NOs:1-2.

Another embodiment relates to a method of producing a soybean plant that has improved resistance to Iron Deficiency Chlorosis as compared to a control plant, wherein the method comprises: (a) isolating a nucleic acid from a soybean plant; (b) detecting in the nucleic acid, the presence of a genetic marker that is associated with improved resistance to Iron Deficiency Chlorosis, wherein said genetic marker is selected from any one or more of SEQ ID NOs:1-2; (c) selecting a first soybean plant based on the presence of the marker associated with improved resistance; (d) crossing a second soybean plant with said first soybean plant, wherein the second soybean plant does not comprise in its genome the marker associated with improved resistance to Iron Deficiency Chlorosis; (e) producing seed from said crossing; and (f) selecting a soybean plant grown from said seed that has improved resistance to Iron Deficiency Chlorosis and comprises the genetic marker associated with improved resistance to Iron Deficiency Chlorosis. Another further embodiment relates to a soybean plant produced by said method, wherein the plant comprises the genetic marker associated with improved resistance to Iron Deficiency Chlorosis.

Another embodiment relates to a method of further comprising the step of backcrossing the plants produced from step (f).

Another embodiment provides for where the selecting comprises marker assisted selection.

In another embodiment, the detecting comprises an oligonucleotide probe. In an embodiment, the method further comprises crossing the one or more plants comprising the indicated resistance to Iron Deficiency Chlorosis to produce one or more F₁ or additional progeny plants, wherein at least one of the F₁ or additional progeny plants comprises the indicated resistance to Iron Deficiency Chlorosis. In another embodiment, the crossing comprises selfing, sibling crossing, or backcrossing. In another embodiment, the selfing, sibling crossing, or backcrossing comprises marker-assisted selection. In another embodiment, the selfing, sibling crossing, or backcrossing comprises marker-assisted selection for at least two generations. In another embodiment, the plant comprises a Glycine max plant.

Another embodiment relates to a Glycine max plant having in its genome, a chromosomal interval, wherein the chromosomal interval comprises detection of resistance to Iron Deficiency Chlorosis beginning at about base pair 36,552,796 and ending at about base pair 36,553,107 on chromosome 3 of the Williams82a2.75 reference genome or equivalent thereof, in other Glycine max lines. Further, said plant comprises any one or more of SEQ ID NOs:1-2 or any portion thereof, conferring resistance to Iron Deficiency Chlorosis or an insertion or deletion marker associated with increased resistance to Iron Deficiency Chlorosis, wherein said insertion or deletion marker corresponds with any one of the markers from any one or more of SEQ ID NOs:1-2.

In another embodiment, the one or more markers comprises an insertion relative to a reference soybean genome for Glycine max at nucleotide position at about base pair 36,552,946 on chromosome 3, wherein the reference genome is the Glycine max reference genome (Williams82a2.75 reference genome).

In another embodiment, the nucleotide position comprises a 12 base pair insertion of CCCGAGTTACAA on soybean chromosome 3 relative to a reference soybean genome, wherein the reference genome is the Glycine max reference genome (Williams82a2.75 reference genome reference genome).

In another embodiment, the one or more markers for detection of resistance to Iron Deficiency Chlorosis on chromosome 3 of the Williams82a2.75 reference genome comprises an insertion of SEQ ID NO:3 at position 36,552,946.

In another embodiment, the nucleotide position comprises an insertion on soybean chromosome 3 relative to a reference soybean genome: CCCGAGTTACAA, wherein the reference genome is the Glycine max Williams82a2.75 reference genome.

### BRIEF DESCRIPTION OF THE SEQUENCE LISTINGS

SEQ ID NO:1 discloses the Glycine max DNA sequence containing the insertion for marker mGLY0011226.

SEQ ID NO:2 discloses the Glycine max DNA sequence containing the deletion for marker mGLY0011226.

SEQ ID NO:3 discloses the Glycine max DNA insertion sequence for marker mGLY0011226.

### DETAILED DESCRIPTION

It is to be understood that the embodiments herein are not limited in their application to the details of construction and the arrangement of the components set forth in the following description. Other embodiments can be practiced or carried out in various ways. Also, it is to be understood that the phraseology and terminology employed herein is for the purpose of description and should not be regarded as limiting.

Throughout this disclosure, various publications, patents and published patent specifications are referenced. Where permissible, the disclosures of these publications, patents and published patent specifications are hereby incorporated by reference in their entirety into the present disclosure to more fully describe the state of the art. Unless otherwise indicated, the disclosure encompasses conventional techniques of plant breeding, immunology, molecular biology, microbiology, cell biology and recombinant DNA, which are within the skill of the art. See, e.g., Sambrook and Russell, Molecular Cloning: A Laboratory Manual, 3rd edition (2001); Current Protocols in Molecular Biology [(F. M. Ausubel, et al. eds., (1987)]; Plant Breeding: Principles and Prospects (Plant Breeding, Vol 1) M. D. Hayward, N, O. Bosemark, I. Romagosa; Chapman & Hall, (1993); Coligan, Dunn, Ploegh, Speicher and Wingfeld, eds. (1995) CURRENT Protocols in Protein Science (John Wiley & Sons, Inc.); the series Methods in Enzymology (Academic Press, Inc.): PCR 2: A Practical Approach (M. J. MacPherson, B. D. Flames and G. R. Taylor eds. (1995)], Harlow and Lane, eds. (1988) Antibodies, A Laboratory Manual, and Animal Cell Culture [R. I. Freshney, ed. (1987)].

Unless otherwise noted, technical terms are used according to conventional usage in the art. Definitions of common terms in molecular biology may be found in Lewin, Genes VII, published by Oxford University Press, 2000; Kendrew et al. (eds.), The Encyclopedia of Molecular Biology, published by Wiley-Interscience, 1999; and Robert A. Meyers (ed.), Molecular Biology and Biotechnology, a Comprehensive Desk Reference, published by VCH Publishers, Inc., 1995; Ausubel et al. (1987) Current Protocols in Molecular Biology, Green Publishing; Sambrook and Russell. (2001) Molecular Cloning: A Laboratory Manual 3rd. edition.

Although the following terms are believed to be well understood by one of ordinary skill in the art, the following definitions are set forth to facilitate understanding of the presently disclosed subject matter.

As used herein, the terms “a” or “an” or “the” may refer to one or more than one. For example, “a” marker (e.g., SNP) can mean one marker or a plurality of markers (e.g., 2, 3, 4, 5, 6, 7, 8, 9, 10 and the like).

As used herein, the term “and/or” refers to and encompasses any and all possible combinations of one or more of the associated listed items, as well as the lack of combinations when interpreted in the alternative (“or”).

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker.

As used herein, the term “Iron Deficiency Chlorosis tolerance/tolerant/resistant” refers to a soybean plant or soybean germplasm that is capable of tolerating iron deficiency and resisting chlorosis of the leaves and stunting of plant growth. When used in reference to germplasm, the term refers to the ability of a plant that arises from that germplasm to tolerate Iron Deficiency Chlorosis. Iron Deficiency Chlorosis may be abbreviated as “IDC”.

The term “favorable” as used herein refers to an allele conferring resistance to Iron Deficiency Chlorosis.

As used herein, the term “soybean” refers to Glycine spp., including Glycine max.

As used herein, the term “detect” or “detecting” refers to any of a variety of methods for determining the presence of a nucleic acid.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, as well as plant parts, such as leaves, stems, pollen, or cells that can be cultured into a whole plant.

As used herein, “haplotype” refers to a series of polymorphisms or gene alleles derived from one parent across one or more loci in the genome (that tend to be inherited together).

The term “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Other examples of such markers are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers, insertion/deletion (InDel) markers, or isozyme markers or combinations of the markers described herein which defines a specific genetic and chromosomal location.

In some embodiments, a genetic marker of an embodiment is a SNP allele and/or combination of SNP alleles (haplotype), and/or polymorphisms which are associated with Iron Deficiency Chlorosis resistance.

“Marker-assisted selection” (MAS) is a process by which phenotypes are selected based on marker genotypes. Marker assisted selection includes the use of marker genotypes for identifying plants for inclusion in and/or removal from a breeding program or planting.

“Tolerance/tolerant/resistant/resistance” is used to describe plants showing some symptoms to for example, to a pest or pathogen, but are still able to produce marketable product with an acceptable yield. Some plants that are referred to as tolerant or resistant are only so in the sense that they may still produce a crop, even though the plants are symptomatic, and the yield may be reduced.

As used herein, the terms “phenotype,” “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait.” In other cases, a phenotype is the result of several genes.

As used herein, the term “plant” includes plant cells, plant protoplasts, plant cells of a tissue culture from which soybean plants can be regenerated, plant callus, plant clumps, and plant cells that are intact in plants or parts of plants, such as pollen, flowers, embryo, seeds, pods, leaves, stems, and the like

As used herein, a “plant part” may be any part of a plant and include a plant cell, or plant tissue.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

The term “single nucleotide polymorphism (SNP)” refers to a change in which a single base in the DNA differs from the usual base at that position. These single base changes are commonly abbreviated as “SNPs”.

The term “substantially identical,” in the context of two nucleic acid or protein sequences, refers to two or more sequences or subsequences that have at least 60%, 80%, 90%, 95%, and at least 99% nucleotide or amino acid residue identity, when compared and aligned for maximum correspondence, as measured using one of the following sequence comparison algorithms or by visual inspection. The substantial identity may exist over a region of the sequences that is at least about 50 residues in length, over a region of at least about 100 residues in length, or over a region of at least about 150 residues in length. In one embodiment, the sequences are substantially identical over the entire length of the coding regions. Furthermore, substantially identical nucleic acid or protein sequences perform substantially the same function.

To determine the percent-identity (“Percent Identity”) between two sequences, in a first step, a pairwise sequence alignment is generated between those two sequences. Pairwise alignments in this first step can be generated by various tools known to a person skilled in the art, like e.g. programs “Blast” (Altschul et al. J. Mol. Biol. 215:403-410), “Blast2” (“gapped Blast”) (Altschul et al., Nucleic Acids Res. 25:3389-3402.), programs from The European Molecular Biology Open Software Suite (EMBOSS, Trends in Genetics 16 (6), 276 (2000)) like “Water”, “Matcher” or “Needle”, or by visual inspection.

After aligning the two sequences, in a second step, a percent-identity value can be determined from the alignment produced. Percent-identity between the two sequences can be calculated from the complete alignment produced, or from a region out of the alignment, e.g. the region of the alignment showing the sequence of one or more embodiments over its complete length, or the region showing the other sequence over its complete length, or from a region showing only parts of the sequences. The alignment region from which a percent-identity value is calculated has a length of at least 100 positions, has a length of at least 150 positions, or has a length of more than 200 positions. For determination of percent-identity, first the sum over all positions is calculated, in which both sequences are showing identical residues in the alignment region, and this sum is then divided by the length of the alignment region, whereby positions in which a sequence has an introduced gap are either component of said length (length of alignment region), or are subtracted from said length (length of alignment region−total number of gaps in alignment region). The obtained value is then multiplied with 100 to result in percent-identity (% identity).

In one embodiment, the two sequences are first aligned over their complete length according the algorithm of Needleman and Wunsch (J. Mol. Biol. (1979) 48, p. 443-453) as implemented in program “Needle” from EMBOSS (Trends in Genetics 16 (6), 276 (2000), preferably version 6.3.1.2 or later with using the programs default parameters (gapopen=10.0, gapextend=0.5 and matrix=EBLOSUM62 (EMBOSS version of the BLOSUM62 substitution)) for protein sequences and default parameters (gapopen=10.0, gapextend=0.5 and matrix=EDNAFULL) for nucleotide sequences. Percent-identity (% identity) is then determined from the complete alignment produced and is calculated as follows: percent-identity=(sum of positions showing identical residues in alignment×100)/(length of alignment−total number of gaps in alignment). This value can also be obtained directly from EMBOSS program “Needle” as program labeled “longest identity” when the parameter option “-nobrief” is applied.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

As used herein, nucleotides are indicated by their bases by the following standard abbreviations: adenine (A), cytosine (C), thymine (T), and guanine (G).

“Quantitative Trait Loci (QTL)” as used herein refers to a region of DNA which is associated with a particular phenotypic trait, which varies in degree and which can be attributed to polygenic effects, i.e., the product of two or more genes, and their environment. These QTLs are often found on different chromosomes.

### Iron Deficiency Chlorosis (IDC)

Soybean is generally planted in areas having a high incidence of soils that are calcareous and have low iron availability. IDC is a direct result of an impaired capacity of the plant to extract soluble iron from the soil, leading to severe leaf chlorosis, low photosynthetic rates, and yield reductions of several million metric tons each year. Iron is required for formation of chlorophyll, the green pigment found in plants. When iron is limiting, iron chlorosis symptoms may be expressed in soybean plants. Interveinal chlorosis is the most common symptom, in which the leaves become yellow, with the veins remaining green. In severe cases, the leaves become white or necrotic, with stunted plant growth. Seed yield loss is a function of the severity of yellowing that occurs to the plant. One of the most common strategies against IDC is to cultivate lines that are naturally less susceptible to iron deficiency. In order to devise breeding and genetic transformation programs that aim at generating high-yielding, IDC-resistant soybean lines, it would be useful to better understand the physiological and molecular mechanisms that enable tolerant plants to survive under iron-limiting conditions. (See Rzodkiewicz, P. et al., “Evaluation of Soybean Varieties for Iron-deficiency Chlorosis” Kansas State University Agricultural Experiment Station and Cooperative Extension Service, January 2006; Vasconcelos, M. W., Li, G. W., Li, C., Grusak, M. A. 2008. Physiology of iron deficiency chlorosis resistance in soybean [abstract]. XIV International Symposium on Iron Nutrition and Interactions in Plants, Oct. 11-16, 2008, Beijing, China. p. 67).

Breeding with Molecular/Genetic Markers

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent.

Thus, genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. Another embodiment provides for the means to identify plants that exhibit resistance to Iron Deficiency Chlorosis by identifying plants having Iron Deficiency Chlorosis specific markers.

In general, MAS (marker assisted selection) uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Genomic selection is another form of marker-assisted selection in which a very large number of genetic markers covering the whole genome are used. With genomic selection, all SNPs are included, each with a different level of effect, in a model to explain the variation of the trait. Genomic selection is based on the analysis of many SNPs, for example tens of thousands or even millions of SNPs. This high number of SNP markers is used as input in a genomic prediction formula that predicts the desired phenotype for MAS.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant are not limited, and can include any combination of markers disclosed herein or any marker linked to the markers disclosed herein, or any markers located within a defined QTL interval.

Similarly, by identifying plants lacking a desired marker locus, plants having unfavorable resistance to Iron Deficiency Chlorosis can be identified and eliminated from subsequent crosses. These marker loci can be introgressed into any desired genomic background, germplasm, plant, line, variety, etc., as part of an overall MAS breeding program designed to enhance resistance to Iron Deficiency Chlorosis.

Thus, another embodiment provides for one skilled in the art to detect the presence or absence of resistance to Iron Deficiency Chlorosis genotypes in the genomes of soybean plants as part of a MAS program, as described herein. In one embodiment, a breeder determines the genotype at one or more markers for a parent having favorable resistance to Iron Deficiency Chlorosis which contains a favorable resistance Iron Deficiency Chlorosis allele, and the genotype at one or more markers for a parent with unfavorable resistance to Iron Deficiency Chlorosis, which lacks the favorable resistance Iron Deficiency Chlorosis allele. A breeder can then reliably track the inheritance of the resistance to Iron Deficiency Chlorosis alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having resistance to Iron Deficiency Chlorosis alleles can be reliably predicted to express the desirable phenotype and progeny that share genotypes with the parent not having resistance to Iron Deficiency Chlorosis alleles can be reliably predicted to express the undesirable phenotype.

### Polymorphisms for Identification of Iron Deficiency Chlorosis Resistance

Polymorphisms include variations in multiple nucleotides at specific locations in the genome. Polymorphisms also include single nucleotide polymorphisms (SNPs), which are variations in a particular single nucleotide that occurs at specific positions in the genome, which are a type of genetic variation among soybean genomes.

### Additional Methods for Detecting Polymorphisms for Marker Assisted Breeding

Polymorphisms in DNA sequences can be detected by hybridization to allele-specific oligonucleotide (ASO) probes as disclosed in U.S. Pat. Nos. 5,468,613 and 5,217,863. The nucleotide sequence of an ASO probe is designed to form either a perfectly matched hybrid or to contain a mismatched base pair at the site of the variable nucleotide residues. The distinction between a matched and a mismatched hybrid is based on differences in the thermal stability of the hybrids in the conditions used during hybridization or washing, differences in the stability of the hybrids analyzed by denaturing gradient electrophoresis or chemical cleavage at the site of the mismatch.

If a SNP creates or destroys a restriction endonuclease cleavage site, it will alter the size or profile of the DNA fragments that are generated by digestion with that restriction endonuclease. As such, plants that possess a variant sequence can be distinguished from those having the original sequence by restriction fragment analysis. SNPs that can be identified in this manner are termed “restriction fragment length polymorphisms” (“RFLPs”). RFLPs have been widely used in human and plant genetic analyses (Glassberg, UK Patent Application 2135774; Skolnick et al., Cytogen. Cell Genet. 32:58-67 (1982); Botstein et al., Ann. J. Hum. Genet. 32:314-331 (1980); Fischer et al., PCT Application WO 90/13668; Uhlen, PCT Application WO 90/11369.

An alternative method of determining SNPs is based on cleaved amplified polymorphic sequences (CAPS) (Konieczny, A. and F. M. Ausubel, Plant J. 4:403-410 (1993); Lyamichev et al., Science 260:778-783 (1993). A modified version of CAPs, known as dCAPs, is a technique for detection of Single Nucleotide Polymorphisms (SNPs). The dCAPS technique introduces or destroys restriction enzyme recognition sites by using primers that containing one or more mismatches to the template DNA. The PCR product modified in this manner is then subjected to restriction enzyme digestion and the presence or absence of the SNP is determined by the resulting restriction pattern. This technique is useful for genotyping known mutations and genetic mapping of isolated DNAs (Neff MM, Neff JD, Chory J, Pepper AE. dCAPS, a simple technique for the genetic analysis of single nucleotide polymorphisms: experimental applications in Arabidopsis thaliana genetics. Plant J. 1998 May; 14(3):387-92).

SNPs can also be identified by single strand conformation polymorphism (SSCP) analysis. The SSCP technique is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5:874-879 (1989). Under denaturing conditions, a single strand of DNA will adopt a conformation that is uniquely dependent on its sequence. This conformation usually will be different even if only a single base is changed. Most conformations have been reported to alter the physical configuration or size sufficiently to be detectable by electrophoresis. A number of protocols have been described for SSCP including, but not limited to Lee et al., Anal. Biochem. 205:289-293 (1992); Suzuki et al., Anal. Biochem. 192:82-84 (1991); Lo et al., Nucleic Acids Research 20:1005-1009 (1992); Sarkar et al., Genomics 13:441-443 (1992).

SNPs may also be detected using a DNA fingerprinting technique called amplified fragment length polymorphism (AFLP), which is based on the selective PCR amplification of restriction fragments from a total digest of genomic DNA to profile that DNA. Vos et al., Nucleic Acids Res. 23:4407-4414 (1995). This method allows for the specific co-amplification of many restriction fragments, which can be analyzed without knowledge of the nucleic acid sequence. AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel (Beismann et al., Mol. Ecol. 6:989-993 (1997); Janssen et al., Int. J. Syst. Bacteriol 47:1179-1187 (1997); Huys et al., Int. J. Syst. Bacteriol. 47:1165-1171 (1997); McCouch et al., Plant Mol. Biol. 35:89-99 (1997); Nandi et al., Mol. Gen. Genet. 255:1-8 (1997); Cho et al. Genome 39:373-378 (1996); Simons et al., Genomics 44:61-70 (1997); Cnops et al., Mol. Gen. Genet. 253:32-41 (1996); Thomas et al., Plant J. 8:785-794 (1995).

SNPs may also be detected using random amplified polymorphic DNA (RAPD) (Williams et al., Nucl. Acids Res. 18:6531-6535 (1990).

SNPs, insertions and deletions can also be detected using KASP (Kompetitive Allele-Specific PCR) assays. KASP is a homogenous, fluorescence-based genotyping variant of polymerase chain reaction. It is based on allele-specific oligo extension and fluorescence resonance energy transfer for signal generation. See, for example, Wilkes, Juliet E., “Development of SNP molecular markers associated with resistance to reniform nematode in soybean using KASP genotyping” Euphytica. Volume 219, article number 27, (2023).

SNPs can be detected by methods as disclosed in U.S. Pat. Nos. 5,210,015; 5,876,930 and 6,030,787 in which an oligonucleotide probe having reporter and quencher molecules is hybridized to a target polynucleotide. The probe is degraded by 5′ to 3′ exonuclease activity of a nucleic acid polymerase.

SNPs can also be detected by labelled base extension methods as disclosed in U.S. Pat. Nos. 6,004,744; 6,013,431; 5,595,890; 5,762,876; and 5,945,283. These methods are based on primer extension and incorporation of detectable nucleoside triphosphates. The primer is designed to anneal to the sequence immediately adjacent to the variable nucleotide which can be detected after incorporation of as few as one labelled nucleoside triphosphate. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labelled sequence-specific oligonucleotide probe.

InDels sites can be identified, with genetic maps constructions and validated using methods well-known in the art. See for example, Wang, Jialan et al. “Development and validation of InDel markers for identification of QTL underlying flowering time in soybean” The Crop Journal. Volume 6, Issue 2, April 2018, Pages 126-135.

Thus, one embodiment relates to a method of determining the genotype of a soybean plant, wherein said method comprises obtaining a sample of nucleic acids from the soybean plant and detecting in the nucleic acids, a plurality of polymorphisms, wherein said plurality of polymorphisms correspond to the insertion or deletion identified in any one or more of SEQ ID NOs:1-2.

### Breeding Methods

There are numerous steps in the development of any desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single cultivar an improved combination of desirable traits from the parental germplasm, such as disease resistance, insect resistance, resistance to drought and heat, and improved agronomic traits. Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

A most difficult task is the identification of individuals that are genetically superior because for most traits the true genotypic value is masked by other confounding plant traits or environmental factors. One method of identifying a superior plant is to observe its performance relative to other experimental lines and widely grown standard cultivars. For many traits a single observation is inconclusive, and replicated observations over time and space are required to provide a good estimate of a line's genetic worth.

The goal of a commercial soybean breeding program is to develop new, unique, and superior soybean cultivars. The breeder initially selects and crosses two or more parental lines, followed by generation advancement and selection, thus producing many new genetic combinations. The breeder can theoretically generate billions of different genetic combinations via this procedure. The breeder has no direct control over which genetic combinations will arise in the limited population size which is grown. Therefore, two breeders will never develop the same line having the same traits.

Each year, the plant breeder selects the germplasm to advance to the next generation. This germplasm is grown under unique and different geographical, climatic, and soil conditions and further selections are then made, during and at the end of the growing season. The lines which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce, with any reasonable likelihood, the same cultivar twice by using the exact same original parents and the same selection techniques. This unpredictability results in the expenditure of large amounts of research monies to develop superior new soybean cultivars.

The complexity of inheritance, the breeding objectives, and the available resources influence the breeding method. Pedigree breeding, recurrent selection breeding, and backcross breeding are breeding methods commonly used in soybean. These methods refer to the manner in which breeding pools or populations are made in order to combine desirable traits from two or more cultivars or various broad-based sources. The procedures commonly used for selection of desirable individuals or populations of individuals are called mass selection, plant-to-row selection, and single seed descent or modified single seed descent. One or a combination of these selection methods can be used in the development of a cultivar from a breeding population.

Introduction of a New Trait or Locus into a Soybean Line

A backcross conversion of a soybean cultivar occurs when DNA sequences are introduced through backcrossing, with a designated soybean cultivar, such as a soybean line having Iron Deficiency Chlorosis resistance, utilized as the recurrent parent. Both naturally occurring and transgenic DNA sequences may be introduced through backcrossing techniques. A backcross conversion may produce a plant with a trait or locus conversion in at least two or more backcrosses, including at least 2 crosses, at least 3 crosses, at least 4 crosses, at least 5 crosses, and the like.

The complexity of the backcross conversion method depends on the type of trait being transferred (single genes or closely linked genes as compared to unlinked genes), the level of expression of the trait, the type of inheritance (cytoplasmic or nuclear), and the types of parents included in the cross. It is understood by those of ordinary skill in the art that for single gene traits that are relatively easy to classify, the backcross method is effective and relatively easy to manage. Desired traits that may be transferred through backcross conversion include, but are not limited to, sterility (nuclear and cytoplasmic), fertility restoration, nutritional enhancements, drought tolerance, nitrogen utilization, altered fatty acid profile, low phytate, industrial enhancements, disease resistance (bacterial, fungal, or viral), insect resistance, and herbicide resistance. In addition, an introgression site itself, such as an FRT site, Lox site, or other site-specific integration site, may be inserted by backcrossing and utilized for direct insertion of one or more genes of interest into a specific plant variety. In some embodiments, the number of loci that may be backcrossed into a soybean cultivar is at least 1, 2, 3, 4, or 5, and/or no more than 6, 5, 4, 3, or 2. The gene or genes for Iron Deficiency Chlorosis resistance, for example, may be used as a selectable marker and/or as a phenotypic trait. A single locus conversion of site-specific integration system allows for the integration of multiple genes at the converted loci.

The backcross conversion may result from either the transfer of a dominant allele or a recessive allele. Selection of progeny containing the trait of interest is accomplished by direct selection for a trait associated with a dominant allele. Transgenes transferred via backcrossing typically function as a dominant single gene trait and are relatively easy to classify. Selection of progeny for a trait that is transferred via a recessive allele requires growing and selfing the first backcross generation to determine which plants carry the recessive alleles. Recessive traits may require additional progeny testing in successive backcross generations to determine the presence of the locus of interest. The last backcross generation is usually selfed to give pure breeding progeny for the gene(s) being transferred, although a backcross conversion with a stably introgressed trait may also be maintained by further backcrossing to the recurrent parent with selection for the converted trait.

Along with selection for the trait of interest, progeny are selected for the phenotype of the recurrent parent. The backcross is a form of inbreeding, and the features of the recurrent parent are automatically recovered after successive backcrosses. Poehlman, Breeding Field Crops, p. 204 (1987). Poehlman suggests from one to four or more backcrosses, but as noted above, the number of backcrosses necessary can be reduced with the use of molecular markers. Other factors, such as a genetically similar donor parent, may also reduce the number of backcrosses necessary. As noted by Poehlman, backcrossing is easiest for simply inherited, dominant, and easily recognized traits.

### Using the Molecular Markers of the Embodiments to Develop New Soybean Varieties Having Iron Deficiency Chlorosis Resistance

The molecular markers of the present embodiments, SEQ ID NOs:1-2, can be used to produce Iron Deficiency Chlorosis resistant soybean plants. Thus, one embodiment is directed to producing a soybean plant having improved resistance to Iron Deficiency Chlorosis as compared to a control plant, which does not have resistance to Iron Deficiency Chlorosis, whereby the method comprises: (a) isolating a nucleic acid from a soybean plant; (b) detecting in the nucleic acid, the presence of a genetic marker that is associated with improved resistance to Iron Deficiency Chlorosis, wherein said genetic marker is selected from any one of or more of SEQ ID NOs:1-2; (c) selecting a first soybean plant based on the presence of the marker associated with improved resistance; (d) crossing a second soybean plant with said first soybean plant, wherein the second soybean plant does not comprise in its genome the marker associated with improved resistance to Iron Deficiency Chlorosis; (e) producing seed from said crossing; and (f) selecting a soybean plant grown from said seed that has improved resistance to Iron Deficiency Chlorosis and comprises the genetic marker associated with improved resistance to Iron Deficiency Chlorosis. Another further embodiment relates to a soybean plant produced by said method, wherein the plant comprises the genetic marker associated with improved resistance to Iron Deficiency Chlorosis. Another embodiment relates to a method of further comprising the step of backcrossing the plants produced from step (f).

The following examples are offered by way of illustration and not by way of limitation.

### Examples

### Iron Deficiency Chlorosis Marker Development and Validation

Iron deficiency chlorosis (IDC) is a significant yield-limiting issue in various soybean production regions worldwide. One of the most effective strategies to safeguard yield potential against IDC is to select soybean varieties that exhibit tolerance to this condition. Previous studies have identified IDC_03 (chromosome 3) as a major QTL associated with this trait. Additionally, a candidate gene, Glyma03g28610, was reported to contain a 12 bp deletion found in iron-inefficient lines.

In the process of marker development and validation for selection at IDC_03, 24 KASP markers flanking or within the gene region were either selected or developed based on a proprietary catalog of soybean variants, including the 12 bp deletion. See for example, Gregory A. Peiffer, Keith E. King, Andrew J. Severin, Gregory D. May, Silvia R. Cianzio, Shun Fu Lin, Nicholas C. Lauter, Randy C. Shoemaker, Identification of Candidate Genes Underlying an Iron Efficiency Quantitative Trait Locus in Soybean, Plant Physiology, Volume 158, Issue 4, April 2012, Pages 1745-1754, for methods well-known in the art for determining marker development. Soybean variants in the region of IDC_03 can be found from public collections ((Song, Qijian, David L. Hyten, Gaofeng Jia, Charles V. Quigley, Edward W. Fickus, Randall L. Nelson, and Perry B. Cregan. 2015. Fingerprinting soybean germplasm and its utility in genomic research, G3: Genesl Genomesl Genetics 50(10):1999-2006.) & Valliyodan, B., Brown, A. V., Wang, J. et al. Genetic variation among 481 diverse soybean accessions, inferred from genomic re-sequencing. Nature. Sci Data 8, 50 (2021) or identified similarly. Select IDC_03 region variant information and flanking sequence is then used to design and procure KASP genotyping assays (Biosearch Technologies. Markers were then used to genotype soybean materials.

Table 2 shows validation results for IDC on chromosome 3 for marker mGLY00112226. Eighty-eight diverse soybean lines scored for IDC were genotyped with the marker. Marker mGLY00112226_C01 demonstrated complete linkage to the IDC trait and 100% accuracy for phenotype prediction, underscoring its utility in distinguishing iron-efficient from iron-inefficient lines. The insertion genotype, is the favorable, or resistant, allele.

TABLE 2
IDC_03 marker validation across
88 soybean samples varying for IDC

| Sample ID | Phenotype | Genotype |
|---|---|---|
| 1 | iron-inefficient | DD |
| 2 | iron-inefficient | DD |
| 3 | iron-inefficient | DD |
| 4 | iron-inefficient | DD |
| 5 | iron-inefficient | DD |
| 6 | iron-inefficient | DD |
| 7 | iron-efficient | II |
| 8 | iron-efficient | II |
| 9 | iron-efficient | II |
| 10 | iron-efficient | II |
| 11 | iron-efficient | II |
| 12 | iron-efficient | II |
| 13 | iron-efficient | II |
| 14 | iron-efficient | II |
| 15 | iron-efficient | II |
| 16 | iron-efficient | II |
| 17 | iron-efficient | II |
| 18 | iron-efficient | II |
| 19 | iron-efficient | II |
| 20 | iron-efficient | II |
| 21 | iron-efficient | II |
| 22 | iron-efficient | II |
| 23 | iron-efficient | II |
| 24 | iron-efficient | II |
| 25 | iron-efficient | II |
| 26 | iron-efficient | II |
| 27 | iron-efficient | II |
| 28 | iron-efficient | II |
| 29 | iron-efficient | II |
| 30 | iron-efficient | II |
| 31 | iron-efficient | II |
| 32 | iron-efficient | II |
| 33 | iron-efficient | II |
| 34 | iron-efficient | II |
| 35 | iron-efficient | II |
| 36 | iron-efficient | II |
| 37 | iron-efficient | II |
| 38 | iron-efficient | II |
| 39 | iron-efficient | II |
| 40 | iron-efficient | II |
| 41 | iron-efficient | II |
| 42 | iron-efficient | II |
| 43 | iron-efficient | II |
| 44 | iron-efficient | II |
| 45 | iron-efficient | II |
| 46 | iron-efficient | II |
| 47 | iron-efficient | II |
| 48 | iron-efficient | II |
| 49 | iron-efficient | II |
| 50 | iron-efficient | II |
| 51 | iron-efficient | II |
| 52 | iron-efficient | II |
| 53 | iron-efficient | II |
| 54 | iron-efficient | II |
| 55 | iron-efficient | II |
| 56 | iron-efficient | II |
| 57 | iron-efficient | II |
| 58 | iron-efficient | II |
| 59 | iron-efficient | II |
| 60 | iron-efficient | II |
| 61 | iron-efficient | II |
| 62 | iron-efficient | II |
| 63 | iron-efficient | II |
| 64 | iron-efficient | II |
| 65 | iron-efficient | II |
| 66 | iron-efficient | II |
| 67 | iron-efficient | II |
| 68 | iron-efficient | II |
| 69 | iron-efficient | II |
| 70 | iron-efficient | II |
| 71 | iron-efficient | II |
| 72 | iron-efficient | II |
| 73 | iron-efficient | II |
| 74 | iron-efficient | II |
| 75 | iron-efficient | II |
| 76 | iron-efficient | II |
| 77 | iron-efficient | II |
| 78 | iron-efficient | II |
| 79 | iron-efficient | II |
| 80 | iron-efficient | II |
| 81 | iron-efficient | II |
| 82 | iron-efficient | II |
| 83 | iron-efficient | II |
| 84 | iron-efficient | II |
| 85 | iron-efficient | II |
| 86 | iron-efficient | II |
| 87 | iron-efficient | II |
| 88 | iron-efficient | II |

All publications and patent applications mentioned in the specification are indicative of the level of skill of those skilled in the art to which the embodiments pertain. All publications and patent applications are herein incorporated by reference to the same extent as if each individual publication or patent application was specifically and individually indicated to be incorporated by reference.

Although the foregoing embodiments have been described in some detail by way of illustration and example for purposes of clarity of understanding, it will be obvious that certain changes and modifications may be practiced within the scope of the appended claims.

That which is claimed:

1. A method of determining the genotype of a soybean plant, wherein said method comprises obtaining a sample of nucleic acids from the soybean plant and detecting in the nucleic acids, a plurality of polymorphisms, wherein said plurality of polymorphisms correspond to the nucleotide polymorphisms identified in any one or more of SEQ ID NOs:1-2.

2. A method for developing an Iron Deficiency Chlorosis resistant soybean plant, wherein the method comprises applying marker enhanced selection to detect one or more polymorphisms, wherein said one or more polymorphisms are selected from the nucleotide polymorphisms in any one or more of SEQ ID NOs:1-2.

3. A method of producing a soybean plant that has improved resistance to Iron Deficiency Chlorosis as compared to a control plant, wherein the method comprises:
a. Isolating a nucleic acid from a soybean plant;
b. Detecting in the nucleic acid, the presence of a genetic marker that is associated with improved resistance to Iron Deficiency Chlorosis, wherein said genetic marker is selected from any one or more of SEQ ID NOs:1-2;
c. Selecting a first soybean plant based on the presence of the marker associated with improved resistance;
d. Crossing a second soybean plant with said first soybean plant, wherein the second soybean plant does not comprise in its genome the marker associated with improved resistance to Iron Deficiency Chlorosis;
e. Producing seed from said crossing; and
f. Selecting a soybean plant grown from said seed that has improved resistance to Iron Deficiency Chlorosis and comprises the genetic marker associated with improved resistance to Iron Deficiency Chlorosis.

4. The method of claim 3, further comprising the step of backcrossing the plants produced from step (f).

5. A soybean plant produced by the method of claim 3, wherein the plant comprises the genetic marker associated with improved resistance to Iron Deficiency Chlorosis.

6. A soybean plant having in its genome, a chromosomal interval, wherein the chromosomal interval comprises detection of resistance to Iron Deficiency Chlorosis beginning at about base pair 36,552,796 and ending at about base pair 36,553,107 on chromosome 3 of the Williams82a2.75 reference genome or equivalent thereof in other Glycine max lines.

7. The plant of any one of claim 7, wherein the chromosome interval comprises:
a. Any one or more of SEQ ID NOs:1-2 or any portion thereof, conferring resistance to Iron Deficiency Chlorosis; or
b. A InDel marker associated with increased resistance to Iron Deficiency Chlorosis, wherein said marker corresponds with any one of the markers from any one or more of SEQ ID NO:1-2.

8. A marker for detecting resistance to Iron Deficiency Chlorosis, wherein said marker comprises a polymorphism relative to a reference soybean genome for Glycine max at nucleotide position 36,552,946 on chromosome 3, wherein the reference genome is the Glycine max Williams82a2.75 reference genome.

9. A marker for detecting resistance to Iron Deficiency Chlorosis, wherein said marker comprises a nucleotide position comprising a polymorphism on soybean chromosome 3 relative to a reference soybean genome of a CCCGAGTTACAA insertion genotype at position 36,552,946, wherein the reference genome is the Glycine max Williams82a2.75 reference genome.


SEQUENCE LISTING (3 sequences)

SEQ ID NO: 1 - DNA, 311 bp, Glycine sp.
  source 1..311: mol_type = genomic DNA
  misc_feature 1..311: note = note = mGLY00112226
  variation 151..162: note = a segment comprising nucleotides positions 151 to 162 can also be absent: the resultant sequence is shown as a separate sequence.
ataaaaagccacaaaatcatactaacaaaaagcaaatgatacttcacctattaaattgccataaatagttatctcattccatgttaattattgttgttgcagaaaaaaatgagcattccgactacagtttcgcgagtcataaaatacatacccgagttacaacagcaagtggaagcactatctaagaaaaaagaggatcttttgtgcagaatttctcggcaattgcaaggagatgcagtgaacaaagattctcaaaggagaatttcccaccacaactctgattttgttgtttcaacaagtaggctcaacga

SEQ ID NO: 2 - DNA, 299 bp, Glycine sp.
  source 1..299: mol_type = genomic DNA
  misc_feature 1..299: note = note = mGLY00112226: with deletion
  variation 150..151: note = a segment of 12 nucleotides (cccgagttacaa) can also be found inserted between residues positions 150 and 151: the resultant sequence is shown as a separate sequence.
ataaaaagccacaaaatcatactaacaaaaagcaaatgatacttcacctattaaattgccataaatagttatctcattccatgttaattattgttgttgcagaaaaaaatgagcattccgactacagtttcgcgagtcataaaatacatacagcaagtggaagcactatctaagaaaaaagaggatcttttgtgcagaatttctcggcaattgcaaggagatgcagtgaacaaagattctcaaaggagaatttcccaccacaactctgattttgttgtttcaacaagtaggctcaacga

SEQ ID NO: 3 - DNA, 12 bp, Glycine sp.
  source 1..12: mol_type = genomic DNA
cccgagttacaa